(19) United States
(12) Patent Application Publication
(10) Pub. No.: US 2015/0088081 A1
(43) Pub. Date: Mar. 26, 2015

(54) Stoma Shield for Ostomy Patients

(71) Applicant: Brian Hakel, Katy, TX (US)

(72) Inventor: Brian Hakel, Katy, TX (US)

(21) Appl. No.: 14/034,900

(22) Filed: Sep. 24, 2013

(51) Int. Cl. A61F 5/445; A61F 5/44

(52) U.S. Cl. CPC A61F 5/445; A61F 5/4408
USPC 604/337

(57) ABSTRACT

A stoma shield for ostomy patients for covering a stoma while permitting increased maneuverability of the patient without cutting off flow within a medical device such as a colostomy bag. The stoma shield for ostomy patients includes a curved upper end and a pair of curved downward extensions extending from its lower end so as to define a curved notch. Any medical device extending from a stoma will pass under the notch so as to allow fluid flow regardless of positioning of the patient. A pair of elongated receiver slots allow connection of a strap to secure the stoma shield to a patient's body and allows the shield to be rotated for a comfortable position.

### CROSS REFERENCE TO RELATED APPLICATIONS

Not applicable to this application.

### STATEMENT REGARDING FEDERALLY SPONSORED RESEARCH OR DEVELOPMENT Not applicable to this application.

### BACKGROUND OF THE INVENTION

1. Field of the Invention

The present invention relates generally to a stoma covering and more specifically it relates to a stoma shield for ostomy patients for covering a stoma while permitting increased maneuverability of the patient without cutting off flow within a medical device such as a colostomy bag.

2. Description of the Related Art

Any discussion of the related art throughout the specification should in no way be considered as an admission that such related art is widely known or forms part of common general knowledge in the field.

Patients often require a stoma after various medical procedures or surgeries. A stoma is generally defined as an opening which connects a portion of a body cavity to the outside environment. Artificial stomas are generally covered or enclosed by various medical devices. For example, in the case of a colostomy, a colostomy bag is secured over the stoma.

It is important that the stoma be covered, such as by a stoma shield. A stoma shield protects a stoma from trauma/impact due to its sensitive, exposed, internal organ. The stoma shield also aids in protection from clothing or straps such as a seat belt. Prior art stoma shields may work well to cover the stoma, but they often provide only limited maneuverability of the patient. This limited maneuverability may result in the medical device being cinched off by the stoma shield, thus resulting in loss of fluid flow.

Comfort may also be significantly compromised by prior art configurations. Contents can back up due to pinching of the shield and add pressure to the ostomy appliance. The appliance adheres to the skin with a series of adhesives, and back pressure can often lead to leaks through the adhesion. The acidic nature of the waste contents can lead to discomfort if leaked, such as skin irritations and complications.

Because of the inherent problems with the related art, there is a need for a new and improved stoma shield for ostomy patients for covering a stoma while permitting increased maneuverability of the patient without cutting off flow within a medical device such as a colostomy bag.

### BRIEF SUMMARY OF THE INVENTION

The invention generally relates to a stoma covering which includes a stoma shield which includes a curved upper end and a pair of curved downward extensions extending from its lower end so as to define a curved notch. Any medical device extending from a stoma will pass under the notch so as to allow fluid flow regardless of positioning of the patient. A pair of elongated receiver slots allow connection of a strap to secure the stoma shield to a patient's body and allows the shield to be rotated for a comfortable position.

There has thus been outlined, rather broadly, some of the features of the invention in order that the detailed description thereof may be better understood, and in order that the present contribution to the art may be better appreciated. There are additional features of the invention that will be described hereinafter and that will form the subject matter of the claims appended hereto. In this respect, before explaining at least one embodiment of the invention in detail, it is to be understood that the invention is not limited in its application to the details of construction or to the arrangements of the components set forth in the following description or illustrated in the drawings. The invention is capable of other embodiments and of being practiced and carried out in various ways. Also, it is to be understood that the phraseology and terminology employed herein are for the purpose of the description and should not be regarded as limiting.

### BRIEF DESCRIPTION OF THE DRAWINGS

Various other objects, features and attendant advantages of the present invention will become fully appreciated as the same becomes better understood when considered in conjunction with the accompanying drawings, in which like reference characters designate the same or similar parts throughout the several views, and wherein:

FIG. 1 is a frontal view of the present invention illustrating its alignment with a stoma medical device.

FIG. 2 is an upper perspective view of the present invention.

FIG. 3 is a rear perspective view of the present invention.

FIG. 4 is a frontal view of the present invention.

FIG. 5 is a top sectional view of the present invention in use.

### DETAILED DESCRIPTION OF THE INVENTION

A. Overview.

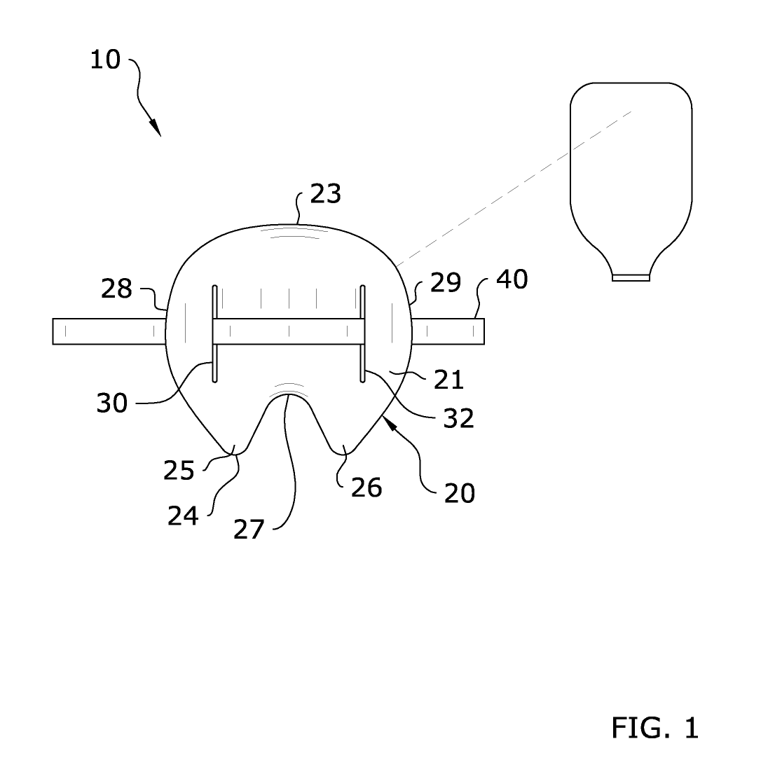

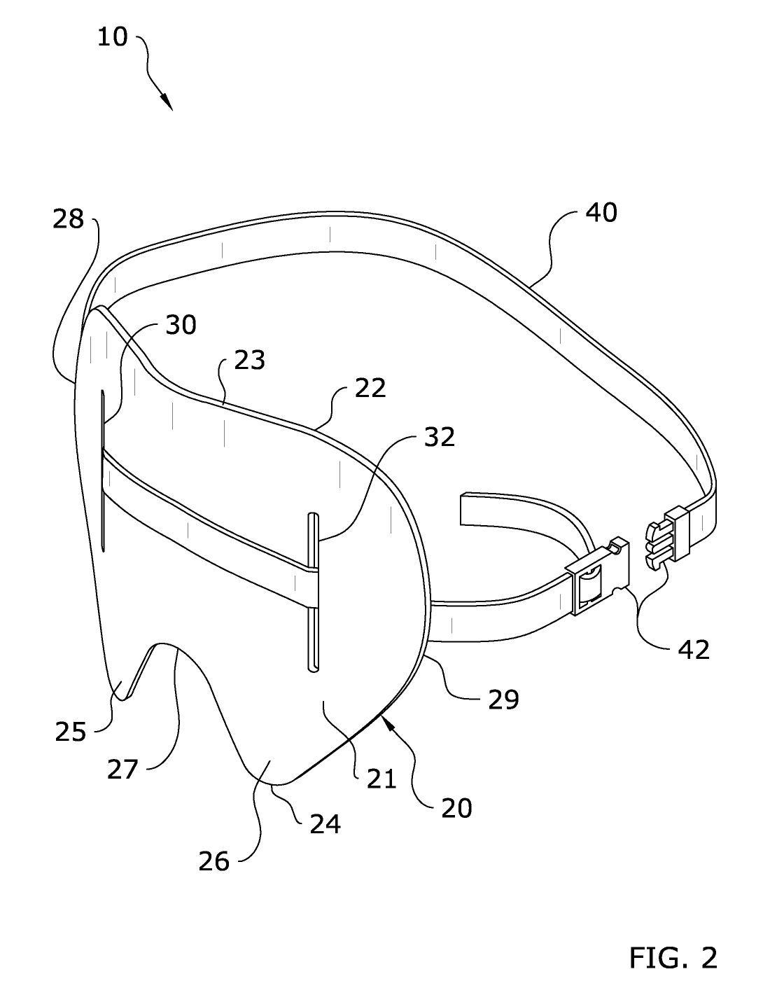

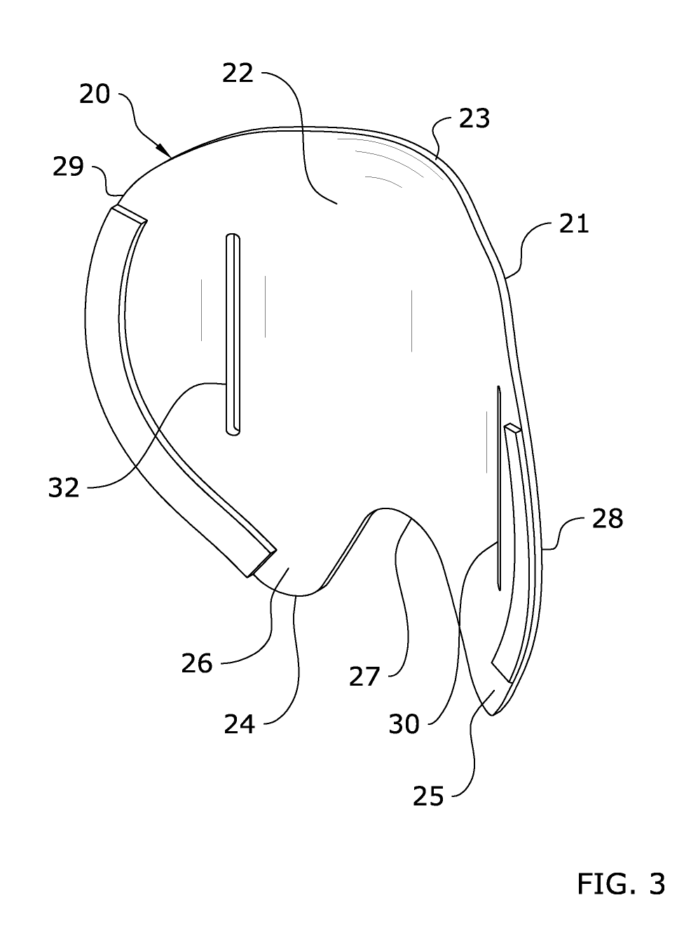

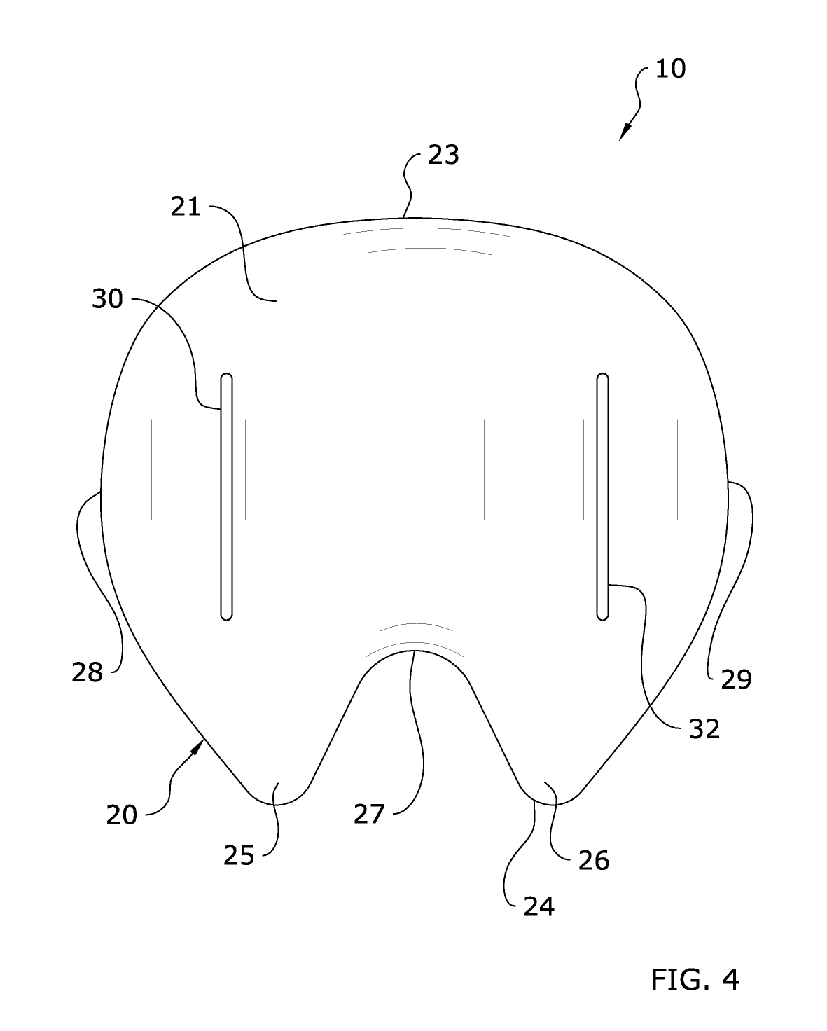

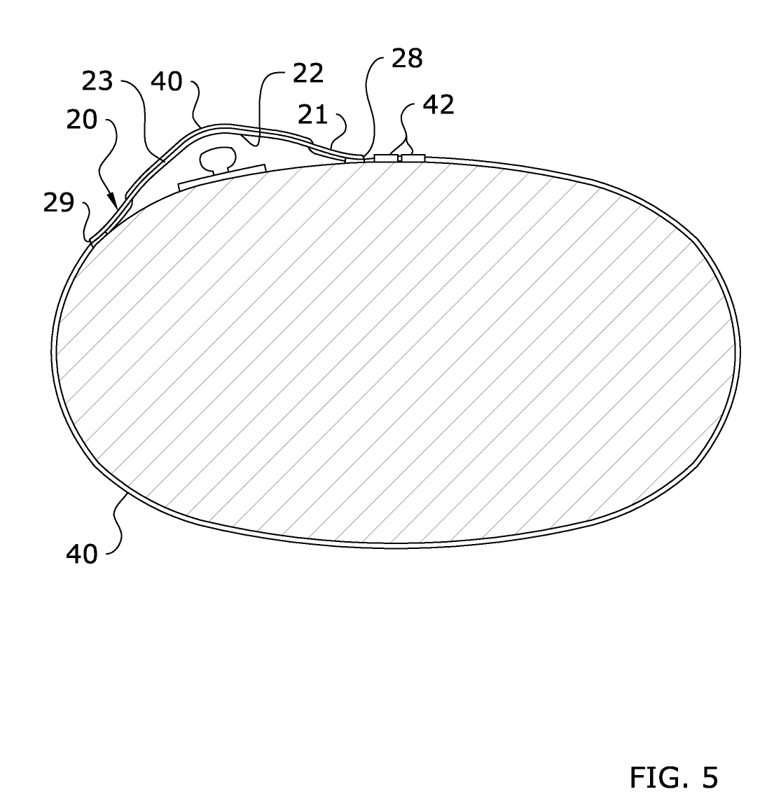

Turning now descriptively to the drawings, in which similar reference characters denote similar elements throughout the several views, FIGS. 1 through 5 illustrate a stoma shield for ostomy patients 10, which comprises a stoma shield 20 which includes a curved upper end 23 and a pair of curved downward extensions 25, 26 extending from its lower end 24 so as to define a curved notch 27. Any medical device extending from a stoma will pass under the notch 27 so as to allow fluid flow regardless of positioning of the patient. A pair of elongated receiver slots 30, 32 allow connection of a strap 40 to secure the stoma shield 20 to a patient's body.

B. Shield.

As shown throughout the figures, the present invention comprises a stoma shield 20 which is adapted for use to cover a stoma as well as any medical device extending therefrom. The stoma shield 20 may be comprised of various materials, but will preferably be comprised of a plastic-type material. Preferably, the stoma shield 20 will be comprised of a resilient material. In some embodiments, the stoma shield 20 may be comprised of a metal or metal alloy.

The stoma shield 20 includes an outer surface 21 and an inner surface 22. The outer surface 21 faces outwardly from the patient. The inner surface 22 of the stoma shield 20 faces inwardly and will act to sandwich any medical device between the shield 20 and the patient's body. The overall body of the shield 20 will preferably be curved inwardly to match the contour of the body as shown in the figures.

The stoma shield 20 includes an upper end 23 which may be slightly curved. The lower end 24 of the stoma shield 20 includes a first extension 25 extending downwardly from a first side 28 of the shield 20 and a second extension 26 extending downwardly from a second side 29 of the shield 20. The extensions 25, 26 are comprised of curved projections which project downwardly from the lower end 24 of the stoma shield 20.

As shown throughout the figures, a notch 27 is defined between the first and second extensions 25, 26 of the stoma shield 20. The notch 27 is allows fluids to pass through the medical device while the patient is sitting or kneeling. In prior stoma shield designs, the stoma shield would cut off flow of fluid through the medical device due to the prior art shield's lower end pinching off the device. The notch 27 prevents such a shortcoming of prior art designs.

The notch 27 will generally extend upwardly and may be comprised of a wide range of shapes and sizes. Preferably, the notch 27 will be comprised of an inverted U- or V-shape as shown in the figures. The shape of the notch 27 may vary depending on the size of the patient, the medical device being covered or the positioning of the stoma.

As shown throughout the figures, a pair of receiver slots 30, 32 extend through the shield 20 from its outer surface 21 to its inner surface 22. A first receiver slot 30 is positioned adjacent to the first side 28 of the shield 20 and a second receiver slot 32 is positioned adjacent to the second side 29 of the shield 20.

The receiver slots 30, 32 are adapted to receive a strap 40 for securing the shield 20 a patient's body. Preferably, the slots 30, 32 will each extend for a length equal to at least half the overall height of the stoma shield 20. The slots 30, 32 will also preferably each be of a length at least three times the width of the strap 40 being used. This allows customization of the strap location height and allows patients to rotate the shield 20 to a more comfortable position. The strap 40 will preferably include one or more connecting members 42 for securing the strap 40 in a closed position, such as a buckle as shown.

C. Operation of Preferred Embodiment.

In use, the shield 20 is positioned over the stoma area. The medical device connected to the stoma, such as a bag, will be positioned to exit from under the shield 20 through the notch 27, which will allow maneuverability without cutting off fluid flow. The strap 40 may be loosened or tightened for a proper fit. The strap 40 may also be adjusted within the slots 30, 32 for increased comfort.

Unless otherwise defined, all technical and scientific terms used herein have the same meaning as commonly understood by one of ordinary skill in the art to which this invention belongs. Although methods and materials similar to or equivalent to those described herein can be used in the practice or testing of the present invention, suitable methods and materials are described above. All publications, patent applications, patents, and other references mentioned herein are incorporated by reference in their entirety to the extent allowed by applicable law and regulations. The present invention may be embodied in other specific forms without departing from the spirit or essential attributes thereof, and it is therefore desired that the present embodiment be considered in all respects as illustrative and not restrictive. Any headings utilized within the description are for convenience only and have no legal or limiting effect.

The invention claimed is:

1. A stoma covering system, comprising:
a shield, said shield including a notch formed within a lower end thereof;
one or more receiver slots formed within said shield; and
a securing member extending through said one or more receiver slots.

2. The stoma covering system of claim 1, wherein said shield is comprised of a curved configuration.

3. The stoma covering system of claim 1, wherein said shield includes a first lower extension and a second lower extension.

4. The stoma covering system of claim 3, wherein said first lower extension is comprised of a curved projection extending downwardly from a point adjacent to a first side of said shield.

5. The stoma covering system of claim 4, wherein said second lower extension is comprised of a curved projection extending downwardly from a point adjacent to a second side of said shield.

6. The stoma covering system of claim 5, wherein said notch is defined between said first lower extension and said second lower extension.

7. The stoma covering system of claim 1, wherein said notch extends upwardly for at least a quarter of a height of said shield.

8. The stoma covering system of claim 1, wherein said notch is comprised of an inverted U-shape.

9. The stoma covering system of claim 1, wherein said one or more receiver slots is comprised of a first receiver slot and a second receiver slot.

10. The stoma covering system of claim 9, wherein said first receiver slot is positioned adjacent to a first side of said shield.

11. The stoma covering system of claim 10, wherein said second receiver slot is positioned adjacent to a second side of said shield.

12. The stoma covering system of claim 9, wherein said first receiver slot and said second receiver slot each extend vertically.

13. The stoma covering system of claim 12, wherein a height of each of said receiver slots is greater than one quarter of a height of said shield.

14. The stoma covering system of claim 1, wherein said securing member is comprised of a strap.

15. The stoma covering system of claim 14, wherein said strap includes a buckle.

16. The stoma covering system of claim 1, wherein said shield is adapted to be positioned over a stoma.

17. The stoma covering system of claim 16, further comprising a medical device secured to said stoma, wherein said medical device extends through said notch.

18. The stoma covering system of claim 1, wherein said shield is comprised of a resilient material.

19. The stoma covering system of claim 18, wherein said shield is comprised of plastic.

20. A stoma covering system, comprising:
a shield adapted to be positioned over a stoma, said shield including a notch formed within a lower end thereof;
a bag secured to said stoma, wherein said bag passes through said notch;
one or more receiver slots formed within said shield; and
a securing member extending through said one or more receiver slots.
